(19) United States
(12) Patent Application Publication
(10) Pub. No.: US 2016/0318860 A1
(43) Pub. Date: Nov. 3, 2016

(54) OXIDATION OF 2-MERCAPTOETHANOL

(71) Applicant: BASF SE, Ludwigshafen (DE)

(72) Inventors: NICOLAS VAUTRAVERS, Mannheim (DE); Joaquim H. TELES, Waldsee (DE)

(21) Appl. No.: 15/102,586

(22) PCT Filed: Dec. 9, 2014
(86) PCT No.: PCT/EP2014/076983

§ 371 Date: Jun. 8, 2016

(30) Foreign Application Priority Data
Dec. 11, 2013 (EP) 13196578.2

(51) Int. Cl. C07C 319/24; C10M 135/24; C07C 323/12

(52) U.S. Cl. CPC C07C 319/24; C07C 323/12; C10M 135/24; C10M 2219/084

(57) ABSTRACT

The present invention relates to a process for the preparation of bis-(2-hydroxyethyl)-disulfide by oxidizing 2-mercaptoethanol with oxygen in a reaction mixture comprising at least one homogeneously distributed iron comprising salt or complex as catalyst and at least one tertiary amine, to bis-(2-hydroxyethyl)-disulfide, obtainable with the process, and to the use of this bis-(2-hydroxyethyl)-disulfide as intermediate in the manufacture of chemical compounds, such as lubricant additives and in the tertiary oil recovery.

The present invention relates to a process for the preparation of bis-(2-hydroxyethyl)-disulfide by oxidizing 2-mercaptoethanol with oxygen in a reaction mixture comprising at least one homogeneously distributed iron comprising salt or complex as catalyst and at least one tertiary amine. The present invention further relates to bis-(2-hydroxyethyl)-disulfide, obtainable with the process according to the present invention and to the use of bis-(2-hydroxyethyl)-disulfide according to the present invention as intermediate in the manufacture of chemical compounds, such as lubricant additives and in the tertiary oil recovery.

Processes for the preparation of bis-(2-hydroxyethyl)-disulfide have already been mentioned in the prior art.

U.S. Pat. No. 4,258,212 and U.S. Pat. No. 5,659,086 describe the oxidation of 2-mercaptoethanol to bis-(2-hydroxyethyl)-disulfide with hydrogen peroxide in the presence of a base. According to U.S. Pat. No. 4,258,212, an alkali metal hydroxide like sodium hydroxide is required to control the pH within a range from 7 to 9. U.S. Pat. No. 5,659,086 teaches the use of an inorganic or organic base, including alkali metal hydroxides like sodium hydroxide as well as primary, secondary or tertiary amines.

A decisive disadvantage of these processes is the use of hydrogen peroxide, due to its high price. Because of its high oxidation capability, hydrogen peroxide also requires careful handling during transport, storage and in particular during its application as oxidizing agent. A further disadvantage of hydrogen peroxide is its high dilution with water. Technical hydrogen peroxide usually has only a content of 50% by weight of H202 for safety reasons. The rest is water, beside traces of a stabilizer, which decreases the efficiency of the oxidation reaction and which leads to a highly diluted reaction product. As consequence the reaction product has to be further processed to obtain the bis-(2-hydroxyethyl)-disulfide with a low water content.

US 2004/0116748 Al discloses the oxidation of mercaptans like 2-mercaptoethanol to the corresponding disulfides by using elemental sulfur as oxidizing agent. A decisive disadvantage of this process is the stoichiometric formation of hydrogen sulfide, which is highly toxic and which has to be removed carefully from the reaction product and disposed.

Y. Wang et al. in Biomacromolecules 2011, 12, 66 to 74, mention the synthesis of bis-(2-hydroxyethyl)disulfide by oxidation of 2-mercaptoethanol using dimethyl sulfoxide as oxidizing agent. A decisive disadvantage of this process is the requirement of dimethyl sulfoxide as oxidation agent, which has to be produced in a complex process. Another decisive disadvantage is the stoichiometric formation of dimethyl sulfide which is a stench and has to be removed from the reaction product and disposed.

S. Murata et al. in Journal of Chemical Society Perkin Transactions, 1989, 617 to 621, disclose the conversion of aromatic nitro compounds with 2-mercaptoethanol in the presence of a iron complex or iron salt and a solvent into the corresponding aromatic amine and bis-(2-hydroxyethyl}-disulfide. A decisive disadvantage of this process is the use of aromatic nitro compounds as the oxidation agent. Aromatic nitro compounds are only accessible through complex processes and are usually toxic or even explosive. Another decisive disadvantage is the stoichiometric formation of the corresponding aromatic amines, which are usually also toxic and which have to be removed from the reaction product and disposed.

Another oxidation agent for oxidizing 2-mercapto ethanol into bis-(2-hydroxyethyl)-disulfide described in the state of the art is oxygen, like pure oxygen or air. The use of oxygen has the general advantage that only small amounts of water are produced as oxidation byproduct.

US 2006/0142616 Al and its German equivalent DE 103 23 839 B3 teach the preparation of bis-(2-hydroxyethyl)-disulfide by oxidation of 2-mercaptoethanol with oxygen in the presence of a copper salt or a manganese salt as catalyst and ammonia or a primary, secondary or tertiary amine as co-catalyst. The conversion is claimed to be nearly 100% without leading to byproducts. According to the teaching of this document, no extensive purification steps are required, but the added ammonia and amines, respectively, are distilled off together with water. Even if the used oxidation agent, the reaction conditions, the conversion and the yield of bis-(2-hydroxyethyl)-disulfide according to this document seem to be beneficial, the applied catalysts show some disadvantages. Copper and manganese are rare, not easy to mine and therefore relatively expensive. Furthermore, copper is well known to be toxic, in particular if absorbed through solid food or fluids like drinking water,

H. Adibi et al. in Chinese Journal of Chemistry, 2008, 26, 2086 to 2092, teach the conversion of 2-mercaptoethanol into bis-(2-hydroxyethyl)-disulfide by oxidation with oxygen in the presence of iron(III) trifiuoroacetate and sodium iodide. The conversion is described to be very high with a yield of 97%. The thiol/catalyst/Nal ratio used in the experiment was 1/0.1/0.2, corresponding to a high concentration of iron(III)trifluoroacetate catalyst of 10 mol-% and a high concentration of sodium iodide of 20 mol-%, whereby both percentage values are in relation to 2-mercaptoethanol.

The object of the present invention is therefore to provide a process for the preparation of bis-(2-hydroxyethyl)-disulfide starting with 2-mercaptoethanol using an oxidant which leaves the smallest possible amount of water or other substances in the crude product. Further, a process shall be provided giving rise to the desired product in high yield and high purity, preferably without additional purification steps. A catalyst shall be used in the process containing at least one metal that is not toxic for animals, human-beings and/or the environment and has therefore not to be separated from the reaction mixture after completion of the reaction.

These objects are solved by the process for the preparation of bis-(2-hydroxyethyl)-disulfide by oxidizing 2-mercaptoethanol with oxygen in a reaction mixture comprising at least one homogeneouosly distributed iron comprising salt or complex as catalyst and at least one tertiary amine, according to the present invention.

The process according to the present invention is conducted to obtain bis-(2-hydroxyethyl)-disulfide by oxidizing 2-mercaptoethanol. The reaction, which is in general known to the skilled artisan, is shown in the following:

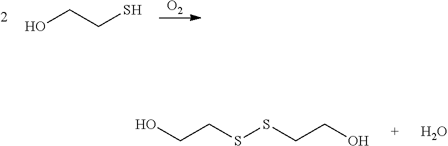

The substrate of the process according to the present invention is 2-mercaptoethanol. It can be prepared using processes that are known to the skilled artisan, for example by the addition of H₂S to ethylene oxide. Further, 2-mercaptoethanol is also commercially available.

In general, 2-mercaptoethanol can be used with a purity that is typical for chemical compounds that are used in chemical reactions. Preferably, 2-mercaptoethanol is used in the process according to the present invention with a purity of at least 95% by weight, more preferably at least 98% by weight.

The desired product that is obtained with the process according to the present invention, is bis-(2-hydroxyethyl)-disulfide and is also in general known to the skilled in the art. According to the present invention, crude bis-(2-hydroxyethyl)-disulfide is in general obtained with a purity of at least 80% by weight, more preferably at least 85° A by weight.

In the process according to the present invention, oxygen is used as the oxidant. According to the present invention oxygen can be added in pure form as a gas. In addition, it is also possible that oxygen is used as a mixture with further gases, preferably with gases that are inert towards the chemical compounds that are present in the reaction mixture according to the present invention.

Suitable gases that may be present in mixtures comprising oxygen that are used according to the present invention are preferably selected from the group consisting of carbon dioxide, noble gases like helium, argon, nitrogen and mixtures thereof.

According to a preferred embodiment of the process according to the present invention, air is used as an oxygen comprising gas. In general, air comprises nitrogen, oxygen, argon, and further gases in minor amounts.

The amount of oxygen that is used in the process according to the present invention is in general adjusted by the pressure of oxygen, in particular by the partial pressure of oxygen in the gas that is used.

In general, the process according to the present invention can be conducted at any partial pressure of oxygen that is suitable, in particular in respect of reaction rate, amount of side products etc.

The process according to the present invention is preferably conducted at a partial pressure of oxygen of 0.2 to 20 bar (a), particularly preferably 1 to 10 bar (a)

The present invention therefore preferably relates to the process according to the present invention, wherein it is conducted at a partial pressure of oxygen of 0.2 to 20 bar (a), particularly preferably 1 to 10 bar (a)

According to the preferred embodiment of the process according to the present invention, wherein air is used as an oxygen comprising gas, the process of the present invention is conducted at a pressure of 1 to 30 bar (a), preferably 5 to 25 bar (a), more preferably 10 to 20 bar (a).

The reaction mixture that is used in the process according to the present invention comprises 2-mercaptoethanol, bis-(2-hydroxyethyl)-disulfide, at least one tertiary amine, at least one iron comprising salt or complex and oxygen.

The present invention therefore preferably relates to the process according to the present invention, wherein the reaction mixtures comprises 2-mercaptoethanol, bis-(2-hydroxyethyl)-disulfide, at least one tertiary amine, at least one iron comprising salt or complex and oxygen.

According to a preferred embodiment of the process according to the present invention, the reaction mixture does not comprise any further components beside substrate, product, catalyst, tertiary amine and oxygen. During the reaction, water is prepared from the oxidant, yielding a reaction mixture that further comprises water in minor amounts. In addition, if air is used as oxygen containing gas, gases like nitrogen and argon are also present in the mixture.

The present invention therefore preferably relates to the process according to the present invention, wherein the reaction mixture consists of 2-mercaptoethanol, bis-(2-hydroxyethyl)-disulfide, at least one tertiary amine, at least one iron comprising salt or complex, water, oxygen and optionally further components like nitrogen and argon, more preferably the reaction mixture consists of 2-mercaptoethanol, bis-(2-hydroxyethyl)-disulfide, at least one tertiary amine, at least one iron comprising salt or complex, water and oxygen.

The present invention therefore preferably relates to the process according to the present invention, wherein the reaction mixture consists of 2-mercaptoethanol, bis-(2-hydroxyethyl)-disulfide, at least one tertiary amine, at least one iron comprising salt or complex, water, oxygen and optionally further components.

Further preferred, the process according to the present invention is conducted in absence of any solvent, preferably in absence of water and/or any organic solvent. According to the present invention, “in absence of any solvent, preferably in absence of water and/or any organic solvent” means that the amount of solvent like water and/or organic solvent is less than 10% by weight, preferably less than 5% by weight.

The process according to the present invention is conducted in the presence of at least one homogeneously distributed iron comprising salt or complex as catalyst.

In general, all iron comprising salts or complexes that are able to be homogeneously distributed in the reaction mixture, may be used. Due to the fact, that the reaction mixture predominantly comprises organic compounds, in particular 2-mercaptoethanol, bis-(2-hydroxyethyl)-disulfide and at least one tertiary amine, the iron salt or complex should be homogeneously distributable in this medium.

Therefore, preferably at least one iron salt or complex is used in the process according to the present invention, having a solubility of at least 0.1 mmol/I, particularly preferably at least 0.2 mmol/I, more preferably at least 0.5 mmol/I, in each case in the above mentioned media and in each case in respect of the whole reaction mixture. An upper limit of the solubility of the at least one iron salt or complex that is used in the process according to the present invention is for example 1.0 moll.

Further preferred, the at least one iron salt or complex is selected from organic or inorganic iron salts or complexes.

The present invention therefore preferably relates to the process according to the present invention, wherein the at least one iron salt or complex is selected from organic or inorganic iron salts or complexes.

Further preferred, the iron being present in the at least one iron salt or complex that is used as catalyst may have any suitable oxidation state like 0, +2 and/or +3, preferably +2 and/or +3.

The present invention therefore preferably relates to the process according to the present invention, wherein the at least one iron salt comprises iron in the oxidation state +2 and/or +3.

Particularly suitable iron salts or complexes, optionally containing at least one neutral ligand, like water, are selected from the group consisting of iron(II) oxide, iron(lll) oxide, iron (II, III) oxide, iron(II) sulphide, iron(ll) disulphide, iron(ll,lll) sulphide, lithium iron(ll) phosphate, lithium iron(III) oxide, iron(ll) phosphide, iron(III) phosphide, iron(III) pyrophosphate, iron(III) phosphate, iron(III) ionophore IV, iron(II) molybdate, ammonium iron(III) hexacyanoferrate(II), iron(lll) ferro-cyanide, 5,10,15,20-Tetrakis(pentafluorophenyI)-21H, 23H-porphyrin iron(III) chloride, 5,10,15,20-Tetraphenyl-21H, 23H-porphine iron(III) chloride, 5,10,15,20-tetrakis(4-methoxyphenyl)-21H, 23H-porphine iron(III) chloride, 2, 3 , 7, 8, 12, 13 , 17, 18-octaethyl-21h,23h-porphine iron(III) acetate, 2,3,7,8,12,13,17,18-Octaethyl-21H,23H-porphine iron(III) chloride, acetyl-cyclopentadienykron(II) carbonyl triphenylphosphine complex, [N,N′-Bis(2-pyridylmethyl)]-2,2′-bipyrrolidinebis(acetonitrile)iron(II) hexafluoroantimonate, iron(II)titanate, iron(II) silicide, iron(III) ionophore VI, bis(cyclopentadienyl) iron(II), cyclopentadienyl(formylcyclopentadienypiron(II), (boronocyclopentadienyl)cyclopentadienyl iron(II), bis(cyclopentadienAiron(III) tetrafluoroborate, cyclopentadienAhydroxymethyl)cyclopentadienyli iron(II), bis(acetylcyclopentadienyl)iron(II), bis(methylcyclopentadienypiron(II), bis(ethylcyclopentadienyl)iron(II), bis(i-propylcyclopentadienypiron(II), dichloro-bis[o-phenylenebis(diphenylphosphine)]iron(II), cyclo-pentadienyliron(II) dicarbonyl iodide, bis(pentamethylcyclopentadienyl)iron(II), bis(tetramethylcyclopentadienyl)iron(II), cyclopentadienyldicarbonyl(tetrahydrofuran)iron(II), tet-rafluoroborate tricarbonyl(cyclooctatetraene)iron(II), cyclopentadienyl iron(II) dicarbonyl dimer, iron(II) hexafluorophosphate, (nicotinamidomethyl)phosphonic acid iron(II) salt, tricarbonyl(2-methoxycyclohexadienylium) iron(II) hexafluorophosphate, tricarbon-yl(4-methoxy-1-methylcyclohexadienylium)iron(II) tetrafluoroborate, meso-tetra(4-N-methypyridyl)porphyrine iron(III), 4-dimethylaminopyridinyl(pentaphenylcyclopentadienyl) iron (II), 4-pyrrolidinopyrindinyl(pentamethylcyclopentadienyl) iron(I), 2,6-Bis41-(2,6-diisopropylphenylimino)ethyljpyridine iron (II) chloride, (aminomethyl)-phosphonic acid, iron(II) salt, bis[(1E)-N-(aminocarbothioyl)ethanehydrazonoyl]iron(II), bis[(III)-N-(anilinocarbothioyl)ethanehydrazonoyl]iron(II), bis[(E)-(aminocarbothioyl)hydrazonol(phenyl)methyl]iron(II), iron(III) i-propoxide, iron(II) acetate, iron(III) oxo acetate perchlorate, ammonium iron(III) citrate, iron(II) acetylacetonate, iron(IU) acetylacetonate, iron(II) bromide, iron(III) bromide, iron(U)chloride. iron(IU)chloride. iron(II) arsenide, iron(III) arsenide, iron(IU)nitrate, iron(II) phthalocyanine bis(pyridine) complex, iron(II) ethylendiammoniumsulfate, iron(II) oxalate, iron(III) oxalate, ammonium iron(III) oxalate, iron(II) fluoride, iron(II)fluoride. iron(II) fumarate, iron(II) gluconate, iron(II) iodide, iron(III) iodide, iron(II) lactate, iron(III) nitrate, iron(II) phthalocyanine, iron(III) phthalocyanine-4, 4′,4″,4′″-tetrasulfonic acid, iron(III) phthalocyanine chloride, iron(II) perchlorate, iron(III) perchlorate, iron(II) sulphate, iron(III) sulphate, ammonium iron(II)sulphate. ammonium iron(UI)sulphate. iron(II) phosphate, iron(III) phosphate, iron(III)tartrate, (+)-iron(II) ascorbate, iron(II)stearate. iron(II) sulfamate, iron(II) tetrafluoroborate, tetraethylammonium tetrachloroiron(III), tris(dibenzoylmethanato)iron(III), tris(ethylenediamine)iron(II) sulfate, iron(III) p-toluenesulfonate, iron(II) trifluoromethanesulfonate, iron(III) trifluoromethanesulfonate, iron(III) trifluoroacetylacetonate, bis(o-phenanthroline)iron(II) cyanide, bis(hexafluoroacetylacetonato)-(N,N,N′,N′-tetrannethylethylenediamine)iron (II), ethylenediaminetetraacetic acid, iron(II1) sodium salt, diethylenetriaminepentaacetic acid iron(II1) disodium salt, bis(N,N′-di-t-butyl acetamidinato)iron(II), iron 2-ethylhexanoate, iron(III) naphthenate, tris(2,2,6,6-tetramethyl-3,5-heptanedionato)iron(III) and mixtures thereof.

The present invention therefore preferably relates to the process according to the present invention, wherein the at least one iron salt or complex, optionally containing at least one neutral ligand, like water, is selected from the group consisting of iron(II) oxide, iron(III) oxide, iron (II, III) oxide, iron(II) sulphide, iron(II) disulphide, iron(II,III) sulphide, lithium iron(II) phosphate, lithium iron(III)oxide. iron(II) phosphide, iron(III) phosphide, iron(III) pyrophosphate, iron(III) phosphate, iron(III) ionophore IV, iron(II) molybdate, ammonium iron(III) hexacyanoferrate(II), iron(III) ferro-cyanide, 5,10,15,20-Tetrakis(pentafluorophenyl)-21H, 23H-porphyrin iron(III) chloride, 5,10,15,20-Tetraphenyl-21H, 23H-porphine iron(III) chloride, 5,10,15,20-tetrakis(4-methoxyphenyI)-21H, 23H-porphine iron(III) chloride, 2, 3 , 7, 8, 12, 13. 17, 18-octaethyl-21h,23h-porphine iron(III) acetate, 2,3,7,8,12,13,17,18-Octaethyl-21H,23H-porphine iron(III) chloride, acetyl-cyclopentadienykron(II) carbonyl triphenylphosphine complex, [N,N′-Bis(2-pyridylmethyl)]-2,2′-bipyrrolidinebis(acetonitrile)iron(II) hexafluoroantimonate, iron(II) titanate, iron(II) silicide, iron(III) ionophore VI, bis(cyclopentadienyl) iron(II), cyclopentadienyl(formylcyclopentadienyl)iron(II), (boronocyclopentadienyl)cyclopentadienyl iron(II), bis(cyclopentadienyl)iron(III) tetrafluoroborate, cyclopentadienyl[hydroxymethyl)cyclopentadienyl] iron(II), bis(acetylcyclopentadienyl)iron(II), bis(methylcyclopentadienypiron(II), bis(ethylcyclopentadienypiron(II), bis(i-propylcyclopentadienypiron(II), dichloro-bis[o-phenylenebis(diphenylphosphine)]iron(II), cyclo-pentadienyliron(II) dicarbonyl iodide, bis(pentamethylcyclopentadienyl)iron(II), bis(tetramethylcyclopentadienyl)iron(II), cyclopentadienyldicarbonyl(tetrahydrofuran)iron(II), tertrafluoroborate tricarbonyl(cyclooctatetraene)iron(II), cyclopentadienyl iron(II) dicarbonyl dimer, cyclopentadienyl(fluorene)iron(II) hexafluorophosphate, (nicotinamidomethyl)phosphonic acid iron(II) salt, tricarbonyl(2-methoxycyclohexadienylium) iron(II) hexafluorophosphate, tricarbon-yl (4-methoxy-1-methylcyclohexadienylium)iron(II) tetrafluoroborate, meso-tetra(4-N-methypyridyl)porphyrine iron(III), 4-dimethylaminopyridinyl(pentaphenylcyclopentadienyl) iron (II), 4-pyrrolidinopyrindinyl(pentamethylcyclopentadienyl) iron(II), 2,6-Bis41-(2,6-diisopropylphenylimino)ethyllpyridine iron (II) chloride, (aminomethyl)-phosphonic acid, iron(II) salt, bis[(IE)-N-(aminocarbothioyl)ethanehydrazonoyl]iron(II), bis[(III)-N-(anilinocarbothioyl)ethanehydrazonoyl]iron(II), bisRE)-(aminocarbothioyl)hydrazono(phenyl)nnethyljiron(II), iron(III) i-propoxide, iron(II) acetate, iron(III) oxo acetate perchlorate, ammonium iron(III) citrate, iron(II) acetylacetonate, iron(III) acetylacetonate, iron(II) bromide, iron(III) bromide, iron(U)chloride. iron(III)chloride. iron(II) arsenide, iron(III) arsenide, iron(III)nitrate. iron(II) phthalocyanine bis(pyridine) complex, iron(II) ethylendiammoniumsulfate, iron(II) oxalate, iron(III) oxalate, ammonium iron(III) oxalate, iron(II) fluoride, iron(III)fluoride_{'} iron(II) fumarate, iron(U)gluconate. iron(II) iodide, iron(IU) iodide, iron(II) lactate, iron(III) nitrate, iron(II) phthalocyanine, iron(III) phthalocyanine-4, 4′,4″,4′″-tetrasulfonic acid, iron(III) phthalocyanine chloride, iron(II) perchlorate, iron(III) perchlorate, iron(II)sulphate. iron(III) sulphate, ammonium iron(II) sulphate, ammonium iron(III) sulphate, iron(II) phosphate, iron(III) phosphate, iron(III) tartrate, (+)-iron(II) ascorbate, iron(II) stearate, iron(II) sulfamate, iron(II) tetrafluoroborate, tetraethylammonium tetrachloroiron(III), tris(dibenzoylmethanato)iron(III), tris(ethylenediamine)iron(II) sulfate, iron(III) p-toluenesulfonate, iron(II) trifluoromethanesulfonate, iron(III) trifluoromethanesulfonate, iron(III) trifluoroacetylacetonate, bis(o-phenanthroline)iron(II) cyanide, bis(hexafluoroacetylacetonato)-(N,N,N′,N′-tetramethylethylenediamine)iron (II), ethylenediaminetetraacetic acid, iron(II1) sodium salt, diethylenetriaminepentaacetic acid iron(III) disodium salt, bis(N,N′-di-t-butyl acetamidinato)iron(II), iron 2-ethylhexanoate, iron(III) naphthenate, tris(2,2,6,6-tetramethyl-3,5-heptanedionato)iron( III) and mixtures thereof.

Particularly preferably, the at least one iron salt or complex is selected from the group consisting of Fe(III)-salts, in particular it is selected from the group consisting of Fe(NO₃)₃. 9 H₂O, Fe₂(SO₄)3, Fe(acetylacetonate)₃, FeCl₃. 6 H₂O and mixtures thereof.

In general the at least one iron salt or complex may be used in any amount which is suitable. Preferably, the catalyst is present in an amount of 0.1 to 50 μmol iron, preferably 1 to 40 μmol iron, particularly preferably 5 to 20 μmol iron, in each case per mol 2-mercaptoethanol.

The present invention therefore preferably relates to the process according to the present invention, wherein the catalyst is present in an amount of 0.1 to 50 μmol iron, preferably 1 to 40 μmol iron, particularly preferably 5 to 20 μmol iron, in each case per mol 2-mercaptoethanol.

The reaction according to the present invention is further conducted in the presence of at least one tertiary amine.

In general, any tertiary amine that is known to the skilled artisan can be used in the process according to the present invention. According to the present invention, the at least one tertiary amine acts as a basic cocatalyst.

According to a preferred embodiment of the present invention, the at least one tertiary amine contains three identical or different, unbranched or branched alkyl radicals having 1 to 20 carbon atoms in each case, where individual carbon atoms can also be, independently of another, replaced by a hetero atom selected from the group consisting of N or O and two or three radicals can also be joined to one another to form a chain comprising at least four atoms.

The present invention therefore preferably relates to the process according to the present invention, wherein the at least one tertiary amine contains three identical or different, unbranched or branched alkyl radicals having 1 to 20 carbon atoms in each case, where individual carbon atoms can also be, independently of another, replaced by a hetero atom selected from the group consisting of N or O and two or three radicals can also be joined to one another to form a chain comprising at least four atoms.

According to a particularly preferred embodiment of the process according to the present invention, the at least one tertiary amine is selected from the group consisting of trimethyl amine, triethyl amine, tripropyl amine, triisopropyl amine, ethyl diisopropyl amine, tri-n-butyl amine, tripentyl amine, trihexyl amine, tricyclohexyl amine, triisoamyl amine, trioctyl amine, tris(2-ethylhexyl) amine, tristearyl amine, trioleyl amine, tridecyl amine, dimethyl stearyl amine, N,N-dimethyl benzyl amine, N,N-dibutyl benzyl amine, N,N-dimethyl aniline, N,N-dihexyl aniline, N,N-diethyl aniline, N,N-dimethyltoluidine, pyridine, quinoline, picoline, 2,4-lutidine, 2,6-lutidine, trimethylpyridine, 2-methyl-5-ethylpyridine (collidine), N-methylpiperidine, N,N-dimethylpiperazine, N-methyl morpholine, N-methyl pyrrolidine, sparteine, tris(2-hydroxyethyl) amine, tris(2-hydroxypropyl) amine, methyl di(2-hydroxyethyl) amine, (N,N-Dimethylaminopropyl)-acetamide, octyldiethyl amine, N-octyl-N-hydroxyethylmethylamine, N,N-didecylmethyl amine, N-dodecyl-N-tetradecylhydroxyethylamine, N,N-ditetradecylmethylamine, N-tetradecyldimethylamine, N-hexadecyl-N-ethylmethylamine, N-octadecyl-N-eicosylmethylamine, N-docosyldimethylamine, N-tetracosyldimethylamine, triethylenediamine, tetramethyl guanidine, DABCO, Pentamethyldiethylenetriamine, N,N,N′,N′-Tetraethyl-1,3-propanediamine, N,N,N′,N′-Tetramethyl-1,4-butanediamine, N,N,N′,N′-Tetramethyl-2-butene-1,4-diamine, N,N,N′,N′-Tetramethyl-1,6-hexanediamine, 1,4,8,11-Tetramethyl-1,4,8,11-tetraazacyclotetradecane, 1,3,5-Trimethylhexahydro-1,3,5-triazine, DBU, DBN and mixtures thereof.

The present invention therefore preferably relates to the process according to the present invention, wherein the at least one tertiary amine is selected from the group consisting of trimethyl amine, triethyl amine, tripropyl amine, triisopropyl amine, ethyl diisopropyl amine, tri-n-butyl amine, tripentyl amine, trihexyl amine, tricyclohexyl amine, trilsoamyl amine, trioctyl amine, tris(2-ethylhexyl) amine, tristearyl amine, trioleyl amine, tridecyl amine, dimethyl stearyl amine, N,N-dimethyl benzyl amine, N,N-dibutyl benzyl amine, N,N-dimethyl aniline, N,N-dihexyl aniline, N,N-diethyl aniline, N,N-dimethyltoluidine, pyridine, quinoline, picoline, 2,4-lutidine, 2,6-lutidine, pyridine. 2-methyl-5-ethylpyridine (collidine), N-methylpiperidine, N,N′-dimethylpiperazine, N-methyl morpholine, N-methyl pyrrolidine, sparteine, tris(2-hydroxyethyl) amine, tris(2-hydroxypropyl) amine, methyl di(2-hydroxyethyl) amine, (N,N-DimethylaminopropyI)-acetamide, octyldiethyl amine, N-octyl-N-hydroxyethylmethylamine, N,N-didecylmethyl amine, N-dodecyl-N-tetradecylhydroxyethylamine, N,N-ditetradecylmethylamine, N-tetradecyldimethylamine, N-hexadecyl-N-ethylmethylamine, N-octadecyl-N-eicosylmethylamine, N-docosyldimethylamine, N-tetracosyldimethylamine, triethylenediamine, tetramethyl guanidine, DABCO, N,N,N′,N″,N″-Pentamethyldiethylenetriamine, N,N,N′,N′-Tetraethyl-1,3-propanediamine, N,N,N′,N′-Tetramethyl-1,4-butanediamine, N,N,N′,N′-Tetramethyl-2-butene-1/4-diamine, N,N,N′,N′-Tetramethyl-1,6-hexanediamine, 1,4,8,11-Tetramethyl-1,4,8,11-tetraazacyclotetradecane, 1,3,5-Trinnethylhexahydro-1,3,5-triazine, DBU, DBN and mixtures thereof.

Most preferably, the at least one tertiary amine is tri-n-butyl-amine,

In general, the at least one tertiary amine may be added in any amount to the reaction mixture according to the present invention, as long as it provides advantages to the process.

According to a preferred embodiment of the process according to the present invention the at least one tertiary amine is added in an amount of 0.01 to 10 mol %, preferably 0.05 to 5 mol %, particularly preferably 0.07 to 3 mol %, in each case based on the amount of 2-mercaptoethanol.

In general, the process according to the present invention can be conducted at any suitable temperature, preferably the process according to the present invention is conducted at a temperature of 0 to 100° C., more preferably 10 to 80° C., particularly preferably 20 to 60° C.

The present invention therefore preferably relates to the process according to the present invention, wherein it is conducted at a temperature of 0 to 100° C. more preferably 10 to 80° C., particularly preferably 20 to 60° C.

The process according to the present invention can be conducted continuously or batchwise.

The process according to the present invention can be conducted in any apparatus known to the skilled artisan and suitable for performing a reaction between a liquid and a gas, like stirred tank reactor, a bubble column or a jet-loop reactor.

The desired product that is obtained from the process according to the present invention can be worked-up, in particular purified, according to any method known to the skilled art, like extraction, distillation etc. According to a preferred embodiment of the process according to the present invention, the product obtained from the process has not to be worked-up, in particular purifled, but can be used directly after being prepared.

The present invention further relates to the bis-(2-hydroxyethyl)-disulfide, obtainable, preferably obtained, with the process according to the present invention. The process according to the present invention gives rise to bis-(2-hydroxyethyl)-disulfide having very specific features, compared to bis-(2-hydroxyethyl)-disulfide obtainable by other processes like low water content and is free of toxic metals. For example, the bis-(2-hydroxyethyl)-disulfide that is obtainable by the process according to the present invention has a preferably low amount of water of less than 15% by weight.

Based on these advantageous properties, bis-(2-hydroxyethyl)-disulfide obtainable, preferably obtained, with the process according to the present invention can be used as intermediate in the manufacture of chemical compounds, such as lubricant additives and in the tertiary oil recovery.

The present invention therefore further relates to the use of bis-(2-hydroxyethyl)-disulfide according to the present invention as intermediate in the manufacture of chemical compounds, such as lubricant additives and in the tertiary oil recovery.

The term “Tertiary Oil recovery” is in general known to the skilled artisan and is a generic term for techniques for increasing the amount of crude oil that can be extracted from an oil field.

Tertiary oil recovery can be accomplished by the injection of various chemicals, usually as dilute solutions. These chemicals are used to aid mobility and the reduction in surface tension. According to the present invention bis-(2-hydroxyethyl)-disulfide is injected to lower the interfacial tension or capillary pressure that impedes oil droplets from moving through a reservoir. According to preferred embodiment, bis-(2-hydroxyethyl)-disulfide is injected into several wells and the production occurs in other nearby wells.

### EXAMPLES

### Examples 1 to 7

### According to the Present Invention

A270 mL autoclave is filledwith 2-mercaptoethanol (1.43 mol), tri-n-butylamine (0.15 mol %) and the respective iron salt (6.5*10⁻⁶ mol Fe per mol 2-mercaptoethanol) and heated at 40° C. under a constant air atmosphere (15 bar). After 24 hours, the reaction mixture is analyzed by ¹H-NMR.

### Example 8

### comparative

The reaction is conducted according to examples 1 to 7 according to the present invention, but without tri-n-butyl amine.

The results of examples 1 to 7 and 8 are shown in the following table:

TABLE 1

| Example | Fe(III) salt | Yield (%) |
|---|---|---|
| 1 | Fe(NO₃)₃•9 H₂O | 91 |
| 2 | Fe₂(SO₄)₃ | 93 |
| 3 | Fe(acetylacetonate)₃ | 95 |
| 4 | FeCl₃•6 H₂O | 95 |
| 5 | FeCl₃•6 H₂O¹⁾ | 98 |
| 6 | FeCl₃•6 H₂O²⁾ | 99 |
| 7 | FeCl₃•6 H₂O³⁾ | 98 |
| 8 (comparative) | FeCl₃•6 H₂O | <10% |

¹⁾1.3 * 10⁻⁵ mol Fe per mol 2-mercaptoethanol and 0.15 mol % of n-tributylamine
²⁾30 bar
³⁾30 bar, 16 hour

### Example 9

### Ccomparative, According to DE 10323839 B3

A 270 mL autoclave is filled with 2-mercaptoethanol (1.43 mol), tri-n-butylamine (0.15 mol %) and MnSO4 (6.5x10⁻⁶ mol Mn per mol 2-mercaptoethanol) and heated at 40° C. under a constant air atmosphere (15 bar). After 24 hours, 97% yield is observed by ¹H-NMR.

1.-13. (canceled)

14. A process for the preparation of bis-(2-hydroxyethyl)-disulfide by oxidizing 2-mercaptoethanol with oxygen in a reaction mixture comprising at least one homogeneously distributed iron comprising salt or complex as catalyst and at least one tertiary amine.

15. The process according to claim 14, wherein the at least one iron salt or complex is an organic or inorganic iron salt or complexes.

16. The process according to claim 14, wherein the at least one iron salt comprises iron in the oxidation state +2 and/or +3.

17. The process according to claim 14, wherein the at least one iron salt or complex, optionally containing at least one neutral ligand and said at least one iron salt or complex is selected from the group consisting of iron(II) oxide, iron(III) oxide, iron (II,III) oxide, iron(II) sulphide, iron(II) disulphide, iron(II,III) sulphide, lithium iron(II) phosphate, lithium iron(III) oxide, iron(II) phosphide, iron(III) phosphide, iron(III) pyrophosphate, iron(III) phosphate, iron(III) ionophore IV, iron(II) molybdate, ammonium iron(III) hexacyanoferrate(II), iron(III) ferrocyanide, 5,10,15,20-Tetrakis(pentafluorophenyl)-21H, 23H-porphyrin iron(III) chloride, 5,10,15,20-Tetraphenyl-21H, 23H-porphine iron(III) chloride, 5,10,15,20-tetrakis(4-methoxyphenyl)-2l H, 23H-porphine iron(III) chloride, 2,3,7,8,12,13,17,18-octaethyl-21h,23h-porphine iron(11I) acetate, 2,3,7,8,12,13,17,18-Octaethyl-21H,23H-porphine iron(III) chloride, acetyl-cyclopentadienyl-iron(II) carbonyl triphenylphosphine complex, [N,N-Bis(2-pyridylmethyl)]-2,2′-bipyrrolidinebis(acetonitrile)iron(II) hexafluoroantimonate, iron(II) titanate, iron(II) silicide, iron(III) ionophore VI, bis(cyclopentadienyl) iron(II), cyclopentadienyl(formylcyclopentadienyl)iron(II), (boronocyclopentadienyl)cyclopentadienyl iron(II), bis(cyclopentadienyl)iron(III) tetrafluoroborate, cyclopentadienyl[(hydroxymethypcyclopentadienyl] iron(II), bis(acetylcyclopentadienyl)iron(II), bis(methylcyclopentadienyl)iron(II), bis(ethylcyclopentadienyl)iron(II), bis(i-propylcyclopentadienyl)iron(II), dichloro-bis[o-phenylenebis(diphenylphosphine)]iron(H), cyclopentadienyliron(II) dicarbonyl iodide, bis(pentamethylcyclopentadienyl)iron(II), bis(tetramethylcyclopentadienyl)iron(II), cyclopentadienyldicarbonyl(tetrahydrofuran)iron(II), tetrafluoroborate tricarbonyl(cyclooctatetraene)iron(II), cyclopentadienyl iron(II) dicarbonyl dimer, cyclopentadienyl(fluorene)iron(II) hexafluorophosphate, (nicotinamidomethyl)phosphonic acid iron(II) salt, tricarbonyl(2-methoxycyclohexadienylium) iron(II) hexafluorophosphate, tricarbonyl(4-methoxy-l-methylcyclohexadienylium)iron(II) tetrafluoroborate, meso-tetra(4-N-methypyridyl)porphyrine iron(III), 4-dimethylaminopyridinyl(pentaphenylcyclopentadienyl) iron (II), 4-pyrrolidinopyrindinyl(pentamethylcyclopentadienyl) iron(II), 2,6-Bis-[1-(2,6-diisopropylphenylimino)ethyl]pyridine iron (II) chloride, (aminomethyl)-phosphonic acid, iron(II) salt, bis[(1E)-N-(aminocarbothioypethanehydrazonoyl]iron(II), bis[(1E)-N-(anilinocarbothioypethanehydrazonoylliron(II), bis[(E)-(aminocarbothioyphydrazono](phenypmethyl]iron(II), iron(III) i-propoxide, iron(II) acetate, iron(III) oxo acetate perchlorate, ammonium iron(III) citrate, iron(II) acetylacetonate, iron(III) acetylacetonate, iron(II) bromide, iron(III) bromide, iron(II) chloride, iron(III) chloride, iron(II) arsenide, iron(III) arsenide, iron(III) citrate, iron(II) phthalocyanine bis(pyridine) complex, iron(II) ethylendiammoniumsulfate, iron(II) oxalate, iron(III) oxalate, ammonium iron(III) oxalate, iron(II) fluoride, iron(III) fluoride, iron(II) fumarate, iron(II) gluconate, iron(II) iodide, iron(III) iodide, iron(II) lactate, iron(III) nitrate, iron(II) phthalocyanine, iron(III) phthalocyanine-4, 4′,4″,4′″-tetrasulfonic acid, iron(III) phthalocyanine chloride, iron(II) perchlorate, iron(III) perchlorate, iron(II) sulphate, iron(III) sulphate, ammonium iron(II) sulphate, ammonium iron(III) sulphate, iron(II) phosphate, iron(III) phosphate, iron(III) tartrate, (+)-iron(II) ascorbate, iron(II) stearate, iron(II) sulfamate, iron(II) tetrafluoroborate, tetraethylammonium tetrachloroiron(III), tris(dibenzoylmethanato)iron(III), tris(ethylenediamine)iron(II) sulfate, iron(III) p-toluenesulfonate, iron(II) trifluoromethanesulfonate, iron(III) trifluoromethanesulfonate, iron(III) trifluoroacetylacetonate, bis(o-phenanthroline)iron(II) cyanide, bis(hexafluoroacetylacetonato)-(N,N,N,Nr-tetramethylethylenediamine)iron (II), ethylenediaminetetraacetic acid, iron(III) sodium salt diethylenetriaminepentaacetic acid iron(III) disodium salt, bis(N,N′-di-t-butyl acetamidinato)iron(II), iron 2-ethylhexanoate, iron(III) naphthenate, tris(2,2,6,6-tetramethyl-3,5-heptanedionato)iron(III) and mixtures thereof.

18. The process according to claim 17, wherein the at least one neutral ligand is water.

19. The process according to claim 14, wherein the catalyst is present in an amount of 0.1 to 50 μmol iron per mol 2-mercaptoethanol.

20. The process according to claim 14, wherein the at least one tertiary amine contains three identical or different, unbranched or branched alkyl radicals having 1 to 20 carbon atoms in each case, where individual carbon atoms can also be, independently of another, replaced by a hetero atom selected from the group consisting of N or O and two or three radicals can also be joined to one another to form a chain comprising at least four atoms.

21. The process according to claim 14, wherein the at least one tertiary amine is selected from the group consisting of trimethyl amine, triethyl amine, tripropyl amine, triisopropyl amine, ethyl diisopropyl amine, tri-n-butyl amine, tripentyl amine, trihexyl amine, tricyclohexyl amine, triisoamyl amine, trioctyl amine, tris(2-ethylhexyl) amine, tristearyl amine, trioleyl amine, tridecyl amine, dimethyl stearyl amine, N,N-dimethyl benzyl amine, N,N-dibutyl benzyl amine, N,N-dimethyl aniline, N,N-dihexyl aniline, N,N-diethyl aniline, N,N-dimethyltoluidine, pyridine, quinoline, picoline, 2,4-lutidine, 2,6-lutidine, trimethylpyridine, 2-methyl-5-ethylpyridine (collidine), N-methylpiperidine, N,N′-dimethylpiperazine, N-methyl morpholine, N-methyl pyrrolidine, sparteine, tris(2-hydroxyethyl) amine, tris(2-hydroxypropyl) amine, methyl di(2-hydroxyethyl) amine, (N,N-Dimethylaminopropyl)-acetamide, octyldiethyl amine, N-octyl-N-hydroxyethylmethylamine, N,N-didecylmethyl amine, N-dodecyl-N-tetradecylhydroxyethylamine, N,N-ditetradecylmethylamine, N-tetradecyldimethylamine, N-hexadecyl-N-ethylmethylamine, N-octadecyl-N-eicosylmethylamine, N-docosyldimethylamine, N-tetracosyldimethylamine, triethylenediamine, tetramethyl guanidine, DABCO, N,N,N′,N″,N″-pentamethyldiethylenetriamine N,N,N,N′-Tetraethyl-1,3-propanediamine, N,N,N′,N′-tetramethyl-1,4-butanediamine, N,N,N′,N′-Tetramethyl-2-butene-1,4-diamine, N,N,N′,N′-Tetramethyl-1,6-hexanediamine, 1,4,8,11-Tetramethyl-1,4,8,11-tetraazacyclotetradecane, 1,3,5-Trimethylhexahydro-1,3,5-triazine, DBU, DBN and mixtures thereof.

22. The process according to claim 14, wherein the reaction mixtures comprises 2-mercaptoethanol, bis-(2-hydroxyethyl)-disulfide, at least one tertiary amine, at least one iron comprising salt or complex and oxygen.

23. The process according to claim 21, wherein the reaction mixture consists of 2-mercaptoethanol, bis-(2-hydroxyethyl)-disulfide, at least one tertiary amine, at least one iron comprising salt or complex, water, oxygen and optionally further components.

24. The process according to claim 14, wherein the process is conducted at a temperature of 0 to 100° C.

25. The process according to claim 14, wherein the process is conducted at a partial pressure of oxygen of 0.2 to 20 bar (a).

26. A bis-(2-hydroxyethyl)-disulfide, obtained by the process according to claim 14.

27. A lubricant additive which comprises the bis-(2-hydroxyethyl)-disulfide according to claim 26.

28. The method of using bis-(2-hydroxyethyl)-disulfide according to claim 25 An intermediate in the manufacture of chemical compounds which comprises the bis-(2-hydroxyethyl)-disulfide according to claim 26.
